(19) United States
(12) Patent Application Publication
(10) Pub. No.: US 2004/0152700 A1
(43) Pub. Date: Aug. 5, 2004

(54) Novel use of 2-phenyl-substituted imidazotriazinones

(76) Inventors: Ulrich Niewohner, Wermelskirchen (DE); Maria Theresia Niewohner, Wermelskirchen (DE); Erwin Bischoff, Wuppertal (DE); Helmut Haning, Wuppertal (DE); Afssaneh Rahbar, Frechen (DE); Tiemo-Jorg Bandel, Oberhausen (DE); Wolfgang Barth, Wuppertal (DE); Franz-Josef Van Der Staay, Dronten (NL)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/476,939

(22) PCT Filed: May 7, 2002
(86) PCT No.: PCT/EP02/05002

(30) Foreign Application Priority Data
May 9, 2001 (DE) 101 22 576.8
May 30, 2001 (DE) 101 26 198.5

(51) Int. Cl. A61K 31/541; A61K 31/5377; A61K 31/53

(52) U.S. Cl. 514/228.5; 514/243; 514/234.5

(57) ABSTRACT

The present invention relates to the use of PDE 5 inhibitors generally and in particular of 2-phenyl-substituted imidazotriazinones for the production of a medicament for improving the perception, concentration power, learning power and/or memory power, in particular for the treatment and/or prophylaxis of disorders of the perception, concentration power, learning power and/or memory power.

The present invention relates to the use of PDE 5 inhibitors generally and in particular of 2-phenyl-substituted imidazotriazinones for the production of a medicament for improving the perception, concentration power, learning power and/or memory power, in particular for the treatment and/or prophylaxis of disturbances of the perception, concentration power, learning power and/or memory power.

The cyclic nucleotide cGMP (cyclic guanosine monophophate) belongs to the most important intracellular messengers and is metabolized by certain phosphodiesterases (PDEs), in particular the isoenzyme PDE 5 (Drugs Fut. 2001, 26, 153-162). PDE 5 occurs especially in vascular smooth muscle tissue, less in the kidney, lung and the blood platelets. On account of their vasorelaxant action, PDE 5 inhibitors have been proposed for the treatment of angina and high blood pressure, but mainly for the treatment of erectile dysfunction.

WO 99/24433 describes 2-phenyl-substituted imidazotriazinones, their cGMP PDE-inhibiting action, and their use for the treatment of vascular disorders, in particular for the treatment of erectile dysfunction.

Behav. Pharmacol. 1999, 10, 731-737 describes the improving action of Sildenafil on the memory capability of mice in an avoidance test.

Surprisingly, it has been found that PDE 5 inhibitors generally and in particular the 2-phenyl-substituted imidazotriazinones described in WO 99/24433 are particularly highly suitable for improving the perception, concentration power, learning power and/or memory power.

Those PDE 5 inhibitors are preferred here which in the test indicated below inhibit PDE 5 with an IC₅₀ of less than 1 μM, preferably less than 0.1 μM.

Preferably, the PDE 5 inhibitors according to the invention are also selective against cAMP PDEs, in particular PDE 4. A 10-fold stronger inhibition of PDE 5 is particularly preferred.

Compounds having inhibitory action on cGMP PDEs are described, for example, in the following specifications: EP-A-0 201 188, EP-A-0 214 708, EP-A-0 293 063, EP-A-0 319 050, EP-A-0 347 027, EP-A-0 347 146, EP-A-0 349 239, EP-A-0 351 058, EP-A-0 352 960, EP-A-0 371 731, EP-A-0 395 328, EP-A-0 400 799, EP-A-0 428 268, EP-A-0 442 204, EP-A-0 463 756, EP-A-0 526 004, EP-A-0 579 496, EP-A-0 607 439, EP-A-0, 640 599, EP-A-0 669 324, EP-A-0 686 625, EP-A-0 722 936, US-A-4,060,615, U.S. Pat. No. 5,294,612, WO 91/19717, WO 94/19351, WO 94/22855, WO 96/32379, WO 97/03070, CAPLUS 1994, 191719 (JP-A-05222000).

Compounds having inhibitory action on the cGMP-specific PDE (corresponds to PDE 5) are described, for example, in the following specifications: EP-A-0 636 626, EP-A-0 668 280, EP-A-0 722 937, EP-A-0 722 943, EP-A-0 722 944, EP-A-0 758 653, EP-A-0 995 750, EP-A-0 995 751, EP-A-1 092 719, WO 94/28902, WO 95/19978, WO 96/16657, WO 96/28159, WO 96/28429, WO 98/49166, WO 99/24433, WO 99/67244, WO 00/78767, WO 01/12608, WO 01/18004, WO 01/19369, WO 01/19802, WO 01/21620, WO 01/27105, J. Med. Chem. 1996, 39, 1635-1644, J. Med Chem. 2000, 43, 1257-1263; Drugs Fut. 2001, 26, 153-162.

Reference is expressly made to the disclosure of these specifications, in particular to the compounds disclosed there.

Preferred compounds used according to the invention are:

a) Sildenafil {5-[2-ethoxy-5-(4-methylpiperazin-1 -ylsulphonyl)phenyl]1,6-dihydro-1-methyl-3-propylpyrazolo[4,3-d]pyrimidin-7-one}, whose preparation is described in EP-A-0 463 756 as Example 12; sildenafil citrate is particularly preferred;

b) IC-351 {(6R, 12aR)-2,3,6,7,12,12a-hexahydro-2-methyl-6-(3,4-methylenedioxy-phenyl)pyrazino[2′,1′:6,1]pyrido[3,4-b]indole-1,4-dione}, whose preparation is described in WO 95/19978 as Examples 78 and 95.

A further aspect of the present invention therefore relates to the use of compounds of the general formula (I)

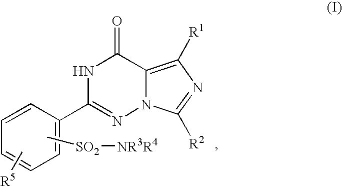

in which

R¹ represents methyl or ethyl,

R² represents ethyl or propyl,

R³ and R⁴ are identical or different and represent a straight-chain or branched alkyl chain having up to 5 carbon atoms, which is optionally substituted up to two times identically or differently by hydroxyl or methoxy,

or

R³ and R⁴ together with the nitrogen atom form a piperidinyl ring, morpholinyl ring, thiomorpholinyl ring or a radical of the formula

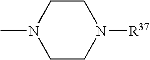

in which

R³⁷ denotes hydrogen, formyl, acyl or alkoxycarbonyl in each case having up to 3 carbon atoms,

or denotes straight-chain or branched alkyl having up to 3 carbon atoms, which is optionally mono- to disubstituted, identically or differently, by hydroxyl, carboxyl, straight-chain or branched alkoxy or alkoxycarbonyl in each case having up to 3 carbon atoms or by groups of the formula -(D)_{f}NR³⁸R³⁹ or —P(O)(OR⁴²)(OR⁴³),

in which

f denotes a number 0 or 1,

D denotes a group of the formula —CO,

R³⁸ and R³⁹ are identical or different and denote hydrogen or methyl,

R⁴² and R⁴³ are identical or different and denote hydrogen, methyl or ethyl,

or

R³⁷ denotes cyclopentyl,

and the heterocycles mentioned under R³ and R⁴, formed together with the nitrogen atom, are optionally mono- to disubstituted, identically or differently, optionally also geminally, by hydroxyl, formyl, carboxyl, acyl or alkoxycarbonyl in each case having up to 3 carbon atoms or groups of the formula —P(O)(OR⁴⁶)(OR⁴⁷) or —(CO)ᵢNR⁴⁹R⁵⁰,

in which

R⁴⁶ and R⁴⁷ are identical or different and denote hydrogen, methyl or ethyl,

i denotes a number 0 or 1,

and

R⁴⁹ and R⁵⁰ are identical or different and denote hydrogen or methyl

and/or the heterocycles mentioned under R³ and R⁴, formed together with the nitrogen atom, are optionally substituted by straight-chain or branched alkyl having up to 3 carbon atoms, which is optionally mono- to disubstituted, identically or differently, by hydroxyl, carboxyl or by a radical of the formula P(O)OR⁵³OR⁵⁴,

in which

R⁵³ and R⁵⁴ are identical or different and denote hydrogen, methyl or ethyl, and/or the heterocycles mentioned under R³ and R⁴, formed together with the nitrogen atom, are optionally substituted by piperidinyl or pyrrolidinyl linked via N,

and

R⁵ represents ethoxy or propoxy,

and their salts, hydrates and/or solvates,

for the production of a medicament for improving the perception, concentration power, learning power and/or memory power.

The compounds used according to the invention can exist in stereoisomeric forms, which either behave as image and mirror image (enantiomers), or which do not behave as image and mirror image (diastereomers). The invention relates both to the enantiomers or diastereomers and to their respective mixtures. The racemic forms, just like the diastereomers, can be separated into the stereoisomerically uniform constituents in a known manner.

The compounds used according to the invention can also be present as salts. Physiologically acceptable salts are preferred in the context of the invention.

Physiologically acceptable salts can be salts of the compounds used according to the invention with inorganic or organic acids. Preferred salts are those with inorganic acids such as, for example, hydrochloric acid, hydrobromic acid, phosphoric acid or sulphuric acid, or salts with organic carboxylic or sulphonic acids such as, for example, acetic acid, maleic acid, fumaric acid, malic acid, citric acid, tartaric acid, lactic acid, benzoic acid, or methanesulphonic acid, ethanesulphonic acid, phenyl-sulphonic acid, toluenesulphonic acid or naphthalenedisulphonic acid.

Physiologically acceptable salts can likewise be metal or ammonium salts of the compounds according to the invention. Those particularly preferred are, for example, sodium, potassium, magnesium or calcium salts, and ammonium salts which are derived from ammonia or organic amines, such as, for example, ethylamine, di- or triethylamine, di- or triethanolamine, dicyclohexylamine, dimethylaminoethanol, arginine, lysine, ethylenediamine or 2-phenylethylamine.

The compounds used according to the invention, in particular the salts, can also be present as hydrates. In the context of the invention, hydrates are understood as meaning those compounds which contain water in the crystal. Such compounds can contain one or more, typically 1 to 5, equivalents of water. Hydrates can be prepared, for example, by crystallizing the compound concerned from water or a water-containing solvent.

Solvates of the compounds according to the invention are stoichiometric compositions of the compounds or their salts with solvents.

An acyl radical having 1 to 3 carbon atoms is, in the context of the invention, for example, formyl, acetyl or ethylcarbonyl.

A straight-chain or branched alkoxy radical having 1 to 3 carbon atoms is, in the context of the invention, methoxy, ethoxy, n-propoxy, or isopropoxy.

An alkoxycarbonyl radical having 1 to 3 carbon atoms is, in the context of the invention, methoxycarbonyl or ethoxycarbonyl.

A straight-chain or branched alkyl radical having 1 to 5 or 1 to 3 carbon atoms is, in the context of the invention, for example, methyl, ethyl, n-propyl, isopropyl, tert-butyl, n-pentyl. Straight-chain or branched alkyl radicals having 1 to 4 or 1 to 3 carbon atoms are preferred.

Halogen in the context of the invention in general represents fluorine, chlorine, bromine and iodine. Fluorine, chlorine and bromine are preferred. Fluorine and chlorine are particularly preferred.

A further embodiment of the invention relates to the use according to the invention of compounds of the general formula (I), in which the radicals R⁵ and —SO₂NR³R⁴ are in the para-position to one another on the phenyl ring and R¹, R², R³, R⁴ and R⁵ have the meaning indicated above.

A further embodiment of the invention relates to the use according to the invention of compounds of the general formula (Ia),

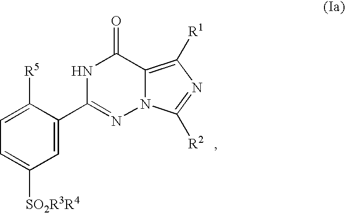

where R¹, R², R³, R⁴ and R⁵ have the meaning indicated above,

and their salts, hydrates and/or solvates.

The use according to the invention of the following compounds is preferred:

2-[2-ethoxy-5-(4-methyl-piperazine-1-sulphonyl)-phenyl]-5,7-dimethyl-3H-imidazo-[5,1-f]-[1,2,4]triazin-4-one;

2-[2-ethoxy-5-(4-hydroxyethylpiperazine-1-sulphonyl)-phenyl]-5,7-dimethyl-3H-imidazo[5,1-f]-[1,2,4]triazin-4-one;

2-[2-ethoxy-5-(4-hydroxypiperidine-1-sulphonyl)-phenyl]-5,7-dimethyl-3H-imidazo-[5,1-f]-[1,2,4]triazin-4-one;

2-[2-ethoxy-5-(4-hydroxymethylpiperidine-1-sulphonyl)-phenyl]-5,7-dimethyl-3H-imidazo[5,1-f][1,2,4]triazin-4-one;

2-[2-ethoxy-5-(3-hydroxypyrrolidine-1-sulphonyl)-phenyl]-5,7-dimethyl-3H-imidazo[5,1-f]-[1,2,4]triazin-4-one;

4-ethoxy-N-ethyl-N-(2-hydroxyethyl)-3-(5,7-dimethyl-4-oxo-3 ,4-dihydro-imidazo-[5,1-f]-[1,2,4]triazin-2-yl)benzenesulphonamide;

N,N-diethyl-4-ethoxy-3-(5,7-dimethyl-4-oxo-3,4-dihydroimidazo[5,1-f][1,2,4]triazin-2-yl)-benzenesulphonamide;

2-[2-ethoxy-5-(4-(2-pyrimidinyl)-piperazine-1-sulphonyl)-phenyl]-5,7-dimethyl-3H-imidazo-[5,1-f][1,2,4]triazin-4-one;

2-[2-ethoxy-5-(morpholine-4-sulphonyl)-phenyl]-5,7-dimethyl-3H-imidazo[5,1-f]-[1,2,4]triazin-4-one;

2-[2-ethoxy-5-(1,4-dioxa-6-azaspiro[4.4]nonane-6-sulphonyl)-phenyl]-5,7-dimethyl-3H-imidazo[5,1-f][1,2,4]triazin-4-one;

N,N-bis-(2-methoxyethyl)-4-ethoxy-3-(5,7-dimethyl-4-oxo-3,4-dihydroimidazo[5,1-f]-[1,2,4]triazin-2-yl)-benzenesulphonamide;

N-(3-isoxazolyl)-4-ethoxy-3-(5,7-dimethyl-4-oxo-3,4-dihydroimidazo[5,1-f][1,2,4]-triazin-2-yl)-benzenesulphonamide;

2-[2-ethoxy-5-(2-t-butoxycarbonylaminomethylmorpholine-4-sulphonyl)-phenyl]-5,7-dimethyl-3H-imidazo[5,1-f][1,2,4]triazin-4-one;

2-[2-ethoxy-5-(4-phenylpiperazine-1-sulphonyl)-phenyl]-5,7-dimethyl-3H-imidazo-[5,1-f]-[1,2,4]triazin-4-one;

2-[2-ethoxy-5-(3-hydroxy-3-methoxymethylpyrrolidine-1-sulphonyl)-phenyl]-5,7-dimethyl-3H-imidazo[5,1-f][1,2,4]triazin-4-one;

2-[2-ethoxy-5-(4-methyl-piperazine-1-sulphonyl)-phenyl]-5-methyl-7-propyl-3H-imidazo[5,1-f][1 ,2,4]triazin-4-one;

2-[2-ethoxy-5-(4-methyl-piperazine-1-sulphonyl)-phenyl]-5-methyl-7-propyl-3H-imidazo[5,1-f][1,2,4]triazin-4-one lactate;

2-[2-ethoxy-5-(4-methyl-piperazine-1-sulphonyl)-phenyl]-5-methyl-7-propyl-3H-imidazo[5,1-f][1,2,4]triazin-4-one hydrochloride;

2-[2-ethoxy-5-(4-ethyl-piperazine-1-sulphonyl)-phenyl]-5-methyl-7-propyl-3H-imidazo[5,1-f][1,2,4]triazin-4-one;

2-[2-ethoxy-5-(4-ethyl-piperazine-1-sulphonyl)-phenyl]-5-methyl-7-propyl-3H-imidazo[5,1-f][1,2,4]triazin-4-one hydrochloride;

2-[2-ethoxy-5-(4-methyl-1-amino-piperazine-1-sulphonyl)-phenyl]-5-methyl-7-propyl-3H-imidazo[5,1-f][1,2,4]triazin-4-one;

2-[2-ethoxy-5-(4-hydroxyethyl-1-amino-piperazine-1-sulphonyl)-phenyl]-5-methyl-7-propyl-3H-imidazo[5,1-f][1,2,4]triazin-4-one;

N,N-bishydroxyethylaminoethyl-4-ethoxy-3-(5-methyl-4-oxo-7-propyl-3,4-dihydro-imidazo[5,1-f][1,2,4]triazin-2-yl)benzenesulphonamide;

2-[2-ethoxy-5 -(4-dimethoxyphosphorylmethyl-piperazine-1-sulphonyl)-phenyl]-5-methyl-7-propyl-3H-imidazo[5,1-f][1,2,4]triazin-4-one;

2-[2-ethoxy-5-(4-diethoxyphosphorylmethyl-piperidine-1-sulphonyl)-phenyl]-5-methyl-7-propyl-3H-imidazo[5,1-f][1,2,4]triazin-4-one;

2-[2-ethoxy-5-(4-hydroxy-piperidine-1-sulphonyl)-phenyl]-5-methyl-7-propyl-3H-imidazo[5,1-f][1,2,4]triazin-4-one;

2-{2-ethoxy-5-[4-(2-hydroxy-ethyl)-piperazine-1-sulphonyl]-phenyl}-5-methyl-7-propyl-3H-imidazo[5,1-f][1,2,4]triazin-4-one;

2-{2-ethoxy-5-[4-(2-hydroxy-ethyl)-piperazine-1-sulphonyl]-phenyl}-5-methyl-7-propyl-3H-imidazo[5,1-f][1,2,4]triazin-4-one hydrochloride;

2-{2-ethoxy-5-[4-(3-hydroxy-propyl)-piperazine-1-sulphonyl]-phenyl}-5-methyl-7-propyl-3H-imidazo[5,1-f][1,2,4]triazin-4-one;

N-allyl-4-ethoxy-N-(2-hydroxy-ethyl)-3-(5-methyl-4-oxo-7-propyl-3,4-dihydro-imidazo[5,1-f][1,2,4]triazin-2-yl)benzenesulphonamide;

N-ethyl-4-ethoxy-N-(2-hydroxy-ethyl)-3-(5-methyl-4-oxo-7-propyl-3,4-dihydro-imidazo[5,1-f][1,2,4]triazin-2-yl)benzenesulphonamide;

N,N-diethyl-4-ethoxy-3-(5-methyl-4-oxo-7-propyl-3,4-dihydro-imidazo[5,1-f]-[1,2,4]triazin-2-yl)benzenesulphonamide;

N-(2-methoxyethyl)-3-(5-methyl-4-oxo-7-propyl-3,4-dihydro-imidazo[5,1-f][1,2,4]-triazin-2-yl)-4-ethoxy-benzenesulphonamide;

N-(2-N,N-dimethylethyl)-3-(5-methyl-4-oxo-7-propyl-3,4-dihydro-imidazo[5,1-f]-[1,2,4]triazin-2-yl)-4-ethoxy-benzenesulphonamide;

N-[3-(1-morpholino)propyl]-3-(5-methyl-4-oxo-7-propyl-3,4-dihydro-imidazo[5,1-f][1,2,4]triazin-2-yl)-4-ethoxy-benzenesulphonamide;

N-{3-[1-(4-methyl)piperazino]-propyl}-3-(5-methyl-4-oxo-7-propyl-3,4-dihydro-imidazo[5,1-f][1,2,4]triazin-2-yl)-4-ethoxy-benzenesulphonamide;

2-{2-ethoxy-5-[4-(2-methoxy-ethyl)-piperazine-1-sulphonyl]-phenyl}-5-methyl-7-propyl-3H-imidazo[5,1-f][1,2,4]triazin-4-one;

2-{2-ethoxy-5-[4-(2-N,N-dimethyl-ethyl)-piperazine-1-sulphonyl]-phenyl}-5-methyl-7-propyl-3H-imidazo[5,1-f][1,2,4]triazin-4-one;

2-{2-ethoxy-5-[4-(3-N,N-dimethyl-propyl)-piperazine-1-sulphonyl]-phenyl}-5-methyl-7-propyl-3H-imidazo[5,1-f][1,2,4]triazin-4-one;

2-[2-ethoxy-5-(4-dioxolano-piperidine-1-sulphonyl)-phenyl]-5-methyl-7-propyl-3H-imidazo[5,1-f][1,2,4]triazin-4-one;

2-[2-ethoxy-5-(4-(5-methyl-4-furoxancarbonyl)-piperazine-1-sulphonyl)-phenyl]-5-methyl-7-propyl-3H-imidazo[5,1-f][1,2,4]triazin-4-one;

2-{2-ethoxy-5-[4-acetyl-piperazine-1-sulphonyl]-phenyl}-5-methyl-7-propyl-3H-imidazo[5,1-f][1,2,4]triazin-4-one;

2-{2-ethoxy-5-[4-formyl-piperazine-1-sulphonyl]-phenyl}-5-methyl-7-propyl-3H-imidazo[5,1-f][1,2,4]triazin-4-one;

2-[2-ethoxy-5-(3-butylsydnoneimine)-1-sulphonyl)-phenyl]-5-methyl-7-propyl-3H-imidazo[5,1-f][1,2,4]triazin-4-one;

5-methyl-2-[5-(4-methyl-piperazine-1-sulphonyl)-2-propoxy-phenyl]-7-propyl-3H-imidazo[5,1-f][1,2,4]triazin-4-one;

5-methyl-2-[5-(4-methyl-piperazine-1-sulphonyl)-2-propoxy-phenyl]-7-propyl-3H-imidazo[5,1-f][1,2,4]triazin-4-one hydrochloride;

2-[5-(4-hydroxypiperidine-1-sulphonyl)-2-propoxy-phenyl]-5-methyl-7-propyl-3H-imidazo[5,1-f][1,2,4]triazin-4-one;

2-[5-(4-hydroxymethylpiperidine-1-sulphonyl)-2-propoxy-phenyl]-5-methyl-7-propyl-3H-imidazo[5,1-f][1,2,4]triazin-4-one;

2-{5-[4-(2-hydroxyethyl)-piperazine-1-sulphonyl]-2-propoxy-phenyl}-5-methyl-7-propyl-3H-imidazo[5,1-f][1,2,4]triazin-4-one;

N-(1,1-dioxotetrahydro-1Δ⁶-thiophen-3-yl)-3-(5-methyl-4-oxo-7-propyl-3,4-dihydro-imidazo-[5,1-f][1,2,4]triazin-2-yl)-4-propoxy-benzenesulphonamide;

N-(2-dimethylaminoethyl)-N-methyl-3-(5-methyl-4-oxo-7-propyl-3,4-dihydro-imidazo[5,1-f][1,2,4]triazin-2-yl)-4-propoxy-benzenesulphonamide;

3-(5-methyl-4-oxo-7-propyl-3,4-dihydro-imidazo[5,1-f][1,2,4]triazin-2-yl)-N-(3-morpholin-4-yl-propyl)-4-propoxy-benzenesulphonamide;

N,N-bis-(2-hydroxyethyl)-3-(5-methyl-4-oxo-7-propyl-3,4-dihydro-imidazo[5,1-f][1,2,4]triazin-2-yl)-4-propoxy-benzenesulphonamide;

N-(3-hydroxybenzyl)-3-(5-methyl-4-oxo-7-propyl-3,4-dihydro-imidazo[5,1-f][1,2,4]-triazin-2-yl)-4-propoxy-benzenesulphonamide;

N-ethyl-N-(2-hydroxyethyl)-3-(5-methyl-4-oxo-7-propyl-3,4-dihydro-imidazo[5,1-f][1,2,4]triazin-2-yl)-4-propoxy-benzenesulphonamide;

N-(3 -ethoxypropyl)-3-(5-methyl-4-oxo-7-propyl-3,4-dihydro-imidazo[5,1-f][1,2,4]-triazin-2-yl)-4-propoxy-benzenesulphonamide;

2-[5(4-hydroxypiperidine-1-sulphonyl)2-propoxy-phenyl]-5-methyl-7-propyl-3H-imidazo[5,1-f][1,2,4]triazin-4-one;

3-(5-methyl-4-oxo-7-propyl-3,4-dihydro-imidazo[5,1-f][1,2,4]triazin-2-yl)-4-prop-oxy-N-pyridin-4-yl-benzenesulphonamide;

N,N-diethyl-3-(5-methyl-4-oxo-7-propyl-3,4-dihydro-imidazo[5,1-f][1,2,4]triazin-2-yl)-4-propoxy-benzenesulphonamide;

1-[3-(5-methyl-4-oxo-7-propyl-3,4-dihydro-imidazo[5,1-f][1,2,4]triazin-2-yl)-4-propoxy-benzenesulphonyl]-piperidine-4-carboxylic acid;

5 -methyl-2-[5-(morpholine-4-sulphonyl)-2-propoxy-phenyl]-7-propyl-3H-imidazo[5,1-f][1,2,4]triazin-4-one;

N-(2-hydroxyethyl)-N-methyl-3-(5-methyl-4-oxo-7-propyl-3,4-dihydro-imidazo[5,1-f][1,2,4]triazin-2-yl)-4-propoxy-benzenesulphonamide;

N-(2-hydroxyethyl)-3-(5-methyl-4-oxo-7-propyl-3 ,4-dihydro-imidazo[5,1-f]-[1,2,4]triazin-2-yl)-4-propoxy-N-propyl-benzenesulphonamide;

N-[2-(3,4-dimethoxy-phenyl)ethyl]-N-methyl-3-(5-methyl-4-oxo-7-propyl-3,4-dihydro-imidazo[5,1-f][1,2,4]triazin-2-yl)-4-propoxy-benzenesulphonamide;

N-allyl-N-(2-hydroxyethyl)-3-(5-methyl-4-oxo-7-propyl-3,4-dihydroimidazo[5,1-f][1,2,4]triazin-2-yl)-4-propoxybenzenesulphonamide;

N-allyl-N-cyclopentyl-3-(5-methyl-4-oxo-7-propyl-3,4-dihydroimidazo[5,1-f]-[1,2,4]triazin-2-yl)-4-propoxybenzenesulphonamide;

N-allyl-N-ethyl-3-(5-methyl-4-oxo-7-propyl-3,4-dihydroimidazo[5,1-f][1,2,4]triazin-2-yl)-4-propoxybenzenesulphonamide;

2-[2-ethoxy-4-methoxy-5-(4-methylpiperazine-1-sulphonyl)-phenyl]-5-methyl-7-propyl-3H-imidazo[5,1-f][1,2,4]triazin-4-one;

2-{2-ethoxy-5-[4-(2-hydroxyethyl)-piperazine-1-sulphonyl]-4-methoxy-phenyl}-5-methyl-7-propyl-3H-imidazo[5,1-f][1,2,4]triazin-4-one;

4-ethoxy-N-ethyl-N-(2-hydroxyethyl)-2-methoxy-5-(5-methyl-4-oxo-7-propyl-3,4-dihydro-imidazo[5,1-f][1,2,4]triazin-2-yl)-benzenesulphonamide;

4-ethoxy-N-(4-ethoxyphenyl)-2-methoxy-5-(5-methyl-4-oxo-7-propyl-3,4-dihydro-imidazo[5,1-f][1,2,4]triazin-2-yl)-benzenesulphonamide;

4-ethoxy-N-ethyl-N-(2-hydroxy-ethyl)-3-(5-ethyl-4-oxo-7-propyl-3,4-dihydroimid-azo[5,1-f][1,2,4]triazin-2-yl)benzenesulphonamide;

N-(2-methoxyethyl)-3-(5-ethyl-4-oxo-7-propyl-3,4-dihydro-imidazo[5,1-f][1,2,4]-triazin-2-yl)-4-ethoxybenzenesulphonamide;

N,N-bis-(2-methoxyethyl)-3-(5-ethyl-4-oxo-7-propyl-3,4-dihydroimidazo[5,1-f]-[1,2,4]triazin-2-yl)-4-ethoxybenzenesulphonamide;

2-[5-(4-hydroxypiperidine-1-sulphonyl)-2-ethoxyphenyl]-5-ethyl-7-propyl-3H-imidazo[5,1-f]-[1,2,4]triazin-4-one;

2-[5-(4-hydroxymethylpiperidine-1-sulphonyl)-2-ethoxy-phenyl]-5-ethyl-7-propyl-3H-imidazo[5,1-f][1,2,4]triazin-4-one;

2-{2-ethoxy-5-[4-(2-hydroxyethyl)-piperazine-1-sulphonyl]-phenyl}-5-ethyl-7-propyl-3H-imidazo[5,1-f][1,2,4]triazin-4-one;

2-[2-ethoxy-5-(4-methylpiperazine-1-sulphonyl)-phenyl]-5-ethyl-7-propyl-3H-imidazo[5,1-f]-[1,2,4]triazin-4-one;

2-[2-ethoxy-5-(4-methylpiperazine-1-sulphonyl)-phenyl]-5-ethyl-7-propyl-3H-imidazo[5,1-f][1,2,4]triazin-4-one hydrochloride;

3-(5-ethyl-4-oxo-7-propyl-3,4-dihydroimidazo[5,1-f][1,2,4]triazin-2-yl)-N-(3-morpholin-4-yl-propyl)-4-ethoxybenzenesulphonamide;

N-(2-hydroxyethyl)-3-(5-ethyl-4-oxo-7-propyl-3,4-dihydro-imidazo[5,1-f][1,2,4]triazin-2-yl)-4-ethoxy-N-propyl-benzenesulphonamide;

2-[2-ethoxy-5-(4-ethyl-piperazine-1-sulphonyl)-phenyl]-5-methyl-7-propyl-3H-imidazo[5,1-f][1,2,4]triazin-4-one hydrochloride trihydrate;

2-[2-ethoxy-5-(4-ethyl-piperazine-1-sulphonyl)-phenyl]-5-methyl-7-propyl-3H-imidazo[5,1-f][1,2,4]triazin-4-one dihydrochloride.

Compounds which are particularly preferably used are mentioned in Table A.

TABLE A
Structure

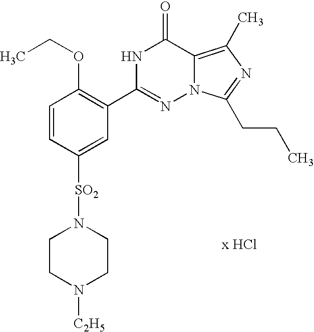

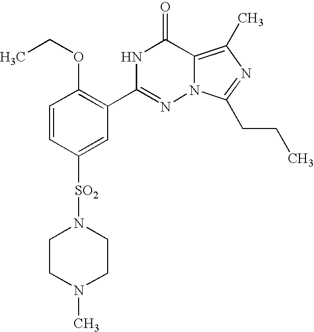

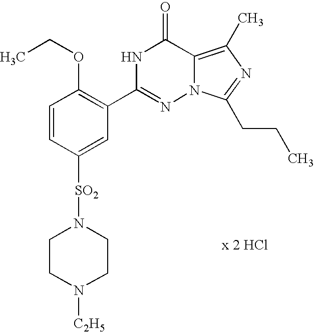

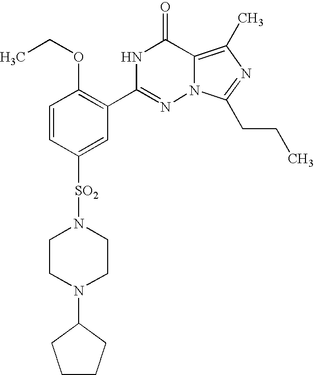

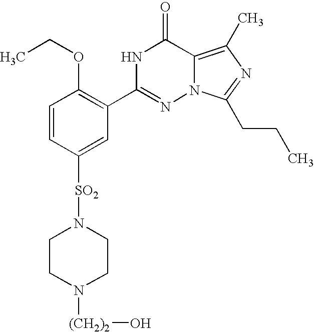

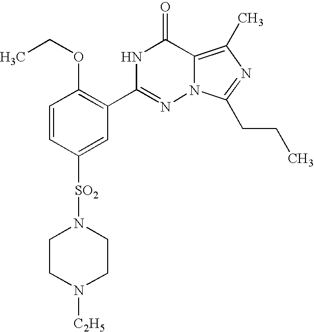

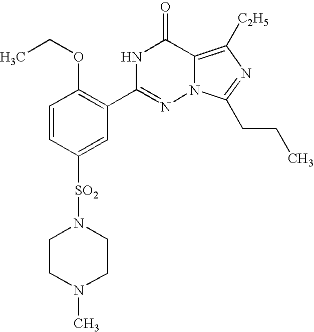

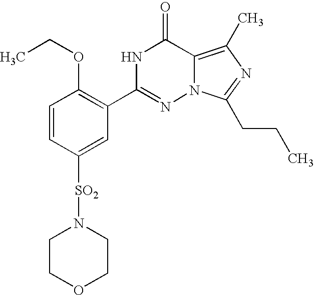

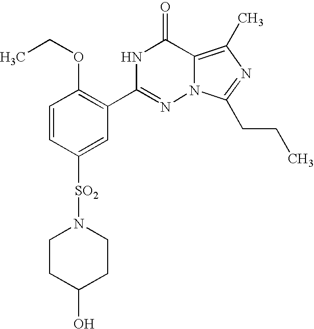

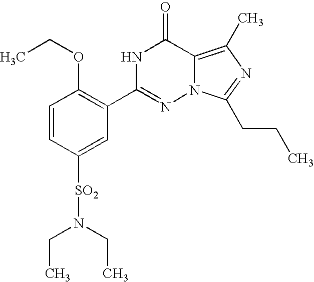

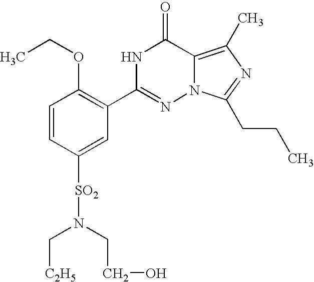

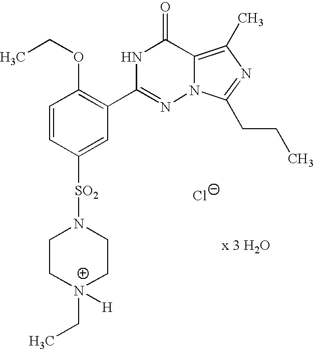

The compounds used according to the invention and their preparation are described in WO 99/24433. Reference is expressly made to the disclosure of WO 99/24433.

A further embodiment of the invention relates to the use of the compounds of the general formula (I) for the production of a medicament for the treatment and/or prophylaxis of disorders of the perception, concentration power, learning power and/or memory power, in particular if the disorder is a result of dementia.

The compounds used according to the invention are particularly suitable for improving the perception, concentration power, learning power or memory power after cognitive disorders, such as occur, in particular, in situations/diseases/syndromes such as “Mild cognitive impairment”, age-associated learning and memory disorders, age-associated memory losses, vascular dementia, craniocerebral trauma, stroke, dementia which occurs after strokes (“post stroke dementia”), post-traumatic craniocerebral trauma, general concentration disorders, concentration disorders in children having learning and memory problems, Alzheimer's disease, vascular dementia, dementia with Lewy bodies, dementia with degeneration of the frontal lobes including Pick's syndrome, Parkinson's disease, progressive nuclear palsy, dementia with corticobasal degeneration, amyolateral sclerosis (ALS), Huntington's disease, multiple sclerosis, thalamic degeneration, Creutzfeld-Jacob's dementia, HIV dementia, schizophrenia with dementia or Korsakoff's psychosis.

The active compound can act systemically and/or locally. For this purpose, it can be administered in a suitable manner, such as, for example, orally, parenterally, pulmonarily, nasally, sublingually, lingually, buccally, rectally, transdermally, conjunctivally, otically or as an implant.

For these administration routes, the active compound can be administered in suitable administration forms.

For oral administration, known administration forms releasing the active compound rapidly and/or in modified form are suitable, such as, for example, tablets (non-coated and coated tablets, e.g. enteric coatings), capsules, sugar-coated tablets, granules, pellets, powders, emulsions, suspensions and solutions.

Parenteral administration can take place with avoidance of an absorption step (intravenous, intra-arterial, intracardiac, intraspinal or intralumbal) or with involvement of an absorption (intramuscular, subcutaneous, intracutaneous, percutaneous, or intraperitoneal). For parenteral administration, suitable administration forms are, inter alia, injection and infusion preparations in the form of solutions, suspensions, emulsions, lyophilizates and sterile powders.

For the other administration routes, for example, pharmaceutical forms for inhalation are suitable, (inter alia powder inhalers, nebulizers), nasal drops/solutions, sprays; tablets or capsules to be administered lingually, sublingually or buccally, suppositories, ear and ophthalmic preparations, vaginal capsules, aqueous suspensions (lotions, shake mixtures), lipophilic suspensions, ointments, creams, milk, pastes, dusting powder or implants.

The active compounds can be converted into the administration forms mentioned in a manner known per se. This is carried out using inert non-toxic, pharmaceutically suitable excipients. These include, inter alia, vehicles (e.g. microcrystalline cellulose), solvents (e.g. liquid polyethylene glycols), emulsifiers (e.g. sodium dodecylsuiphate), dispersing agents (e.g. polyvinylpyrrolidone), synthetic and natural biopolymers (e.g. albumin), stabilizers (e.g. antioxidants such as ascorbic acid), colourants (e.g. inorganic pigments such as iron oxides) or taste and/or odour corrigents.

In general, it has proved advantageous, in the case of parenteral administration, to administer amounts of approximately 0.001 to 30 mg/kg, preferably approximately 0.01 to 10 mg/kg, of body weight to achieve efficacious results. In the case of oral administration, the amount is approximately 0.01 to 100 mg/kg, preferably approximately 0.1 to 30 mg/kg, of body weight.

In spite of this, if appropriate it may be necessary to depart from the amounts mentioned, namely depending on the body weight, route of administration, individual behaviour towards the active compound, type of preparation and time or interval at which administration takes place.

### EXAMPLE 1

is 2-[2-ethoxy-5-(4-methyl-piperazine-1-sulphonyl)-phenyl]-5-methyl-7-propyl-3H-imidazo[5,1-f][1,2,4]triazin-4-one and is prepared according to Example 16 in WO 99/24433.

### EXAMPLE 2

is 2-[2-ethoxy-5-(4-ethyl-piperazine-1-sulphonyl)-phenyl]-5-methyl-7-propyl-3H-imidazol[5,1-f][1,2,4]triazin-4-one hydrochloride trihydrate and is prepared according to Example 336 in WO 99/24433.

The PDE 5-inhibiting action of the compounds used according to the invention can be determined as follows.

Activity of the PDE 5

To test the inhibiting action, the “phosphodiesterase [³H] cGMP-SPA enzyme assay” of Amersham Life Science is used. The test is carried out according to the experimental protocol specified by the manufacturer. Human recombinant PDE 5 is used which has been expressed in a Baculovirus system. The substance concentration is measured at which the reaction rate is reduced by 50%.

Inhibition of the PDEs 1-5 and 7

Recombinant PDE1C (GenBank/EMBL Accession Number: NM_{—}005020), PDE2A (Rosman et al. Gene 1997, 191, 89-95), PDE3B (Miki et al. Genomics 1996, 36, 476-485), PDE4B (Bolger et al. Mol. Cell. Biol. 1993, 13, 6558-6571), PDE5A (GenBank/EMBL Accession Number: AJ004865) and PDE7B (Hetman et al. Proc. Natl. Acad. Sci. U.S.A. 2000, 97, 472-476) were expressed in Sf9 cells with the aid of the pFASTBAC Baculovirus expression system (GibcoBRL).

The in vitro action of test substances on recombinant PDE3B, PDE4B and PDE7B is determined according to the test protocol described above for PDE 10A. For the determination of a corresponding action on recombinant PDE1C, PDE2A and PDE5A, the protocol is adapted as follows: in the case of PDE1C, calmodulin 10⁻⁷ M and CaCl₂ 3 mM are additionally added to the reaction batch. PDE2A is stimulated in the test by addition of cGMP 1 μM and tested using a BSA concentration of 0.01%. For PDE5A, the substrate employed is [8-³H] guanosine 3′, 5′-cyclic phosphate (Amersham Pharmacia Biotech., Piscataway, N.J.).

Examples 1 and 2 show IC₅₀ values of 0.6 and 0.7 nM in the PDE 5 test.

The action according to the invention of the substances can be shown in vivo, for example, in the object recognition test.

Object Recognition Test

The object recognition test is a memory test. It measures the ability of rats (and mice) to differentiate between known and unknown objects.

The test was carried out as described (Blokland et al. NeuroReport 1998, 9, 4205-4208; Ennaceur, A., Delacour, J., Behav. Brain Res. 1988, 31, 47-59; Ennaceur, A., Meliani, K., Psychopharmacology 1992, 109, 321-330; Prickaerts, et al. Eur. J. Pharmacol. 1997, 337, 125-136).

In a first passage, a rat is confronted with two identical objects in an otherwise empty relatively large observation arena. The rat will investigate, i.e. sniff and touch, both objects thoroughly. In a second passage, after an interval of 24 hours, the rat is again placed in the observation arena. One of the known objects is now replaced by a new, unknown object. If a rat recognizes the known object, it will especially investigate the unknown object. After 24 hours, however, a rat has normally forgotten which object it has already investigated in the first passage, and will therefore inspect both objects to the same extent. The administration of a substance having learning- and memory-improving action will lead to a rat recognizing the object already seen 24 hours beforehand, in the first passage, as known. It will investigate the new, unknown object in more detail than the already known one. This memory power is expressed in a discrimination index. A discrimination index of zero denotes that the rat has investigated both objects, the old one and the new one, for an equally long time; i.e. it has not recognized the old object and reacts to both objects as if they were unknown and new. A discrimination index of greater than zero denotes that the rat has inspected the new object for longer than the old one; i.e. the rat has recognized the old object.

The effects of Example 1 on the object recognition of rats 24 hours after the first passage were investigated. The animals received oral Tylose on its own, or Example 1 in the doses 1.0, 3.0 or 10 mg/kg of body weight, suspended in Tylose, immediately following the first passage using two identical objects. 24 hours later in each case, the second passage followed. After a wash-out period of 2 or 3 days, a new dose of Example 1 was tested in the same rats until the memory power of all rats had been determined twice in all doses. All animals thus served as their own control. The results of this study are shown in FIG. 2. Surprisingly, the memory power in the second passage after treatment with 1.0, 3.0 or 10 mg/kg of Example 1 was improved compared with the control condition (treatment with Tylose on its own). The discrimination index was greater than zero and differed from the discrimination index achieved in the control condition.

1. Use of PDE 5 inhibitors for the production of a medicament for improving the perception, concentration power, learning power and/or memory power.

2. Use of compounds of the general formula (I), 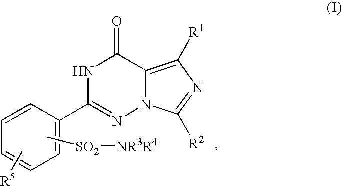
in which
R¹ represents methyl or ethyl,
R² represents ethyl or propyl,
R³ and R⁴ are identical or different and represent a straight-chain or branched alkyl chain having up to 5 carbon atoms, which is optionally substituted up to two times identically or differently by hydroxyl or methoxy,
or
R³ and R⁴ together with the nitrogen atom form a piperidinyl ring, morpholinyl ring, thiomorpholinyl ring or a radical of the formula 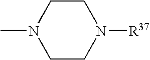
in which
R³⁷ denotes hydrogen, formyl, straight-chain or branched acyl or alkoxycarbonyl in each case having up to 3 carbon atoms,
or denotes straight-chain or branched alkyl having up to 3 carbon atoms, which is optionally mono- to disubstituted, identically or differently, by hydroxyl, carboxyl, straight-chain or branched alkoxy or alkoxycarbonyl in each case having up to 3 carbon atoms or by groups of the formula -(D)_{f}NR³⁸R³⁹ or —P(O)(OR⁴²)(OR⁴³),
in which
f denotes a number 0 or 1,
D denotes a group of the formula —CO,
R³⁸ and R³⁹ are identical or different and denote hydrogen or methyl,
R⁴² and R⁴³ are identical or different and denote hydrogen, methyl or ethyl,
or
R³⁷ denotes cyclopentyl,
and the heterocycles mentioned under R³ and R⁴, formed together with the nitrogen atom, are optionally mono- to disubstituted, identically or differently, optionally also geminally, by hydroxyl, formyl, carboxyl,
acyl or alkoxycarbonyl in each case having up to 3 carbon atoms or groups of the formula —P(O)(OR⁴⁶)(OR⁴⁷) or —(CO)ᵢNR⁴⁹R⁵⁰,
in which
R⁴⁶ and R⁴⁷ are identical or different and denote hydrogen, methyl or ethyl,
i denotes a number 0 or 1,
and
R⁴⁹ and R⁵⁰ are identical or different and denote hydrogen or methyl
and/or the heterocycles mentioned under R³ and R⁴, formed together with the nitrogen atom, are optionally substituted by straight-chain or branched alkyl having up to 3 carbon atoms, which is optionally mono- to disubstituted, identically or differently, by hydroxyl, carboxyl or by a radical of the formula P(O)OR⁵³OR⁵⁴,
in which
R⁵³ and R⁵⁴ are identical or different and denote hydrogen, methyl or ethyl,
and/or the heterocycles mentioned under R³ and R⁴, formed together with the nitrogen atom, are optionally substituted by piperidinyl or pyrrolidinyl linked via N,
and
R⁵ represents ethoxy or propoxy,
and their salts, hydrates and/or solvates,
for the production of a medicament for improving the perception, concentration power, learning power and/or memory power.

3. Use according to claim 1 of compounds of the general formula (Ia), 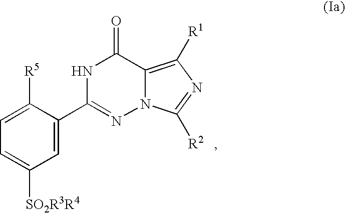
where R¹, R², R³, R⁴ and R⁵ have the meaning indicated in claim 1,
and their salts, hydrates and/or solvates.

4. Use according to claim 1, where the compounds are selected from the group having the following structures:
Structure

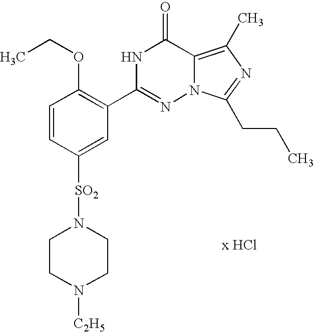

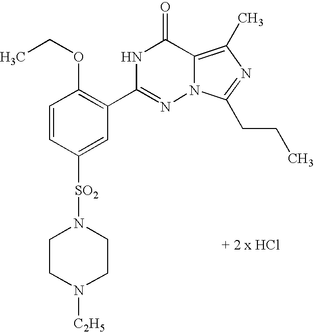

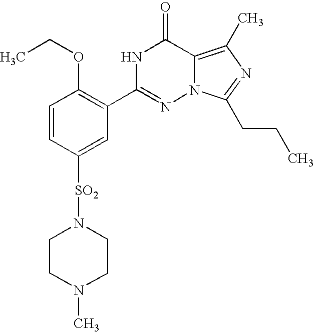

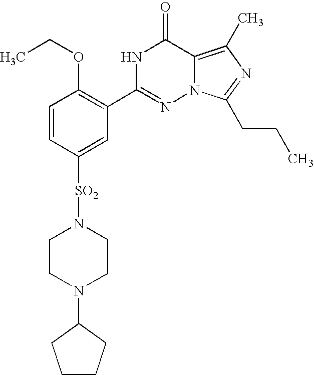

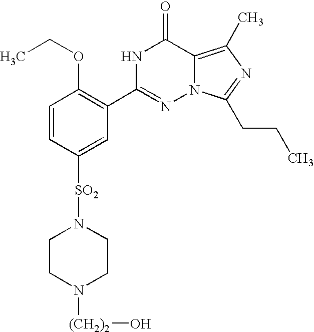

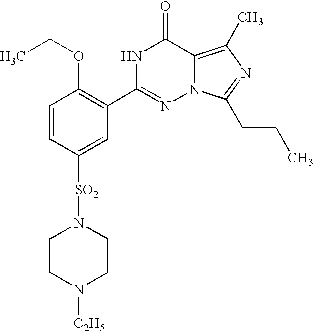

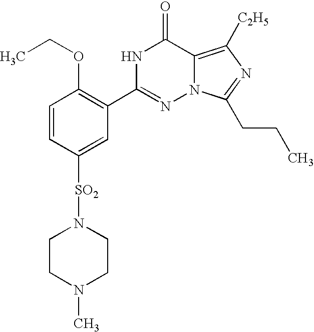

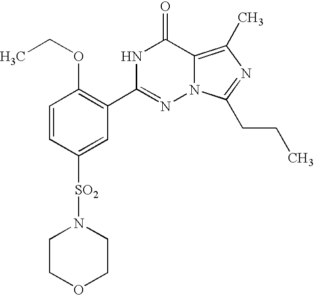

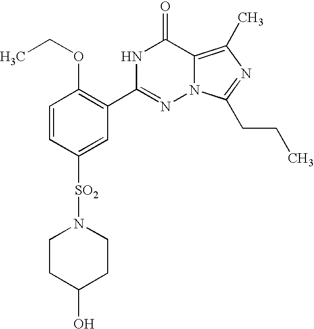

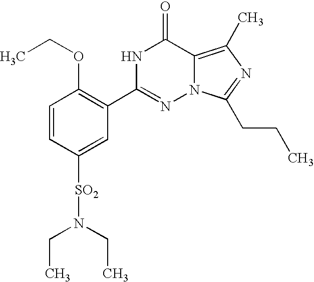

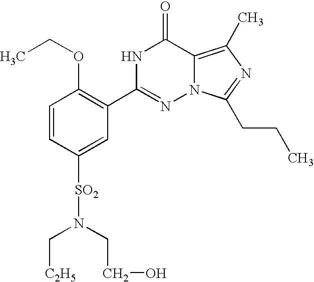

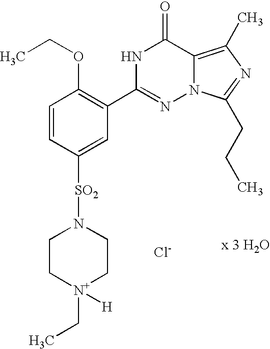

5. Use of compounds according to one of claims 1 to 3 for the production of a medicament for the treatment and/or prophylaxis of disorders of the perception, concentration power, learning power and/or memory power.

6. Use according to claim 4, where the disorder is a result of dementia.
